(19) United States
(12) Plant Patent Application Publication
(10) Pub. No.: US 2008/0184420 P1
(43) Pub. Date: Jul. 31, 2008

(54) Sedum plant named 'Diamond Edge'

(76) Inventor: Robbert Gijsbertus Cornelis Van Blokland, AJ De Hoef (NL)

Correspondence Address: JONDLE & ASSOCIATES P.C., 858 HAPPY CANYON ROAD SUITE 230, CASTLE ROCK, CO, 80108, US

(21) Appl. No.: 11/504,959

(22) Filed: Aug. 16, 2006

(51) Int. Cl. A01H 5/00

(52) U.S. Cl. PLT/263

(57) ABSTRACT

A new Sedum plant particularly distinguished by greenish-yellow variegated foliage, is disclosed.

### GENUS AND SPECIES

Sedum

### VARIETY DENOMINATION

‘Diamond Edge’

### BACKGROUND OF THE NEW PLANT

The present invention comprises a new and distinct cultivar of Sedum, botanically known as Sedum, and hereinafter referred to by the cultivar name ‘Diamond Edge’. The new cultivar originated from a naturally occurring mutation in the Sedum plant ‘Matrona’ (unpatented) found in 2001 in a nursery in Zwaanshoek, The Netherlands.

The new cultivar was created in 2001 in Zwaanshoek, The Netherlands and has been asexually reproduced repeatedly by vegetative cuttings and tissue culture in Zwaanshoek, The Netherlands over a four-year period. The present invention has been found to retain its distinctive characteristics through successive asexual propagations.

Plant Breeder's Rights for this cultivar were applied for with the European Union on Apr. 3, 2006.

### SUMMARY OF THE INVENTION

The most outstanding and distinguishing characteristic of this new cultivar when grown under normal horticultural practices in Zwaanshoek, The Netherlands is greenish-yellow variegated foliage.

### DESCRIPTION OF PHOTOGRAPHS

This new Sedum plant is illustrated by the accompanying photographs which show overall foliage of the plant; the colors shown are as true as can be reasonably obtained by conventional photographic procedures. The photographs are of two-year-old plants grown outside in De Hoef, The Netherlands in June 2006.

FIG.1 shows the overall foliage.

FIG. 2 shows a leaf.

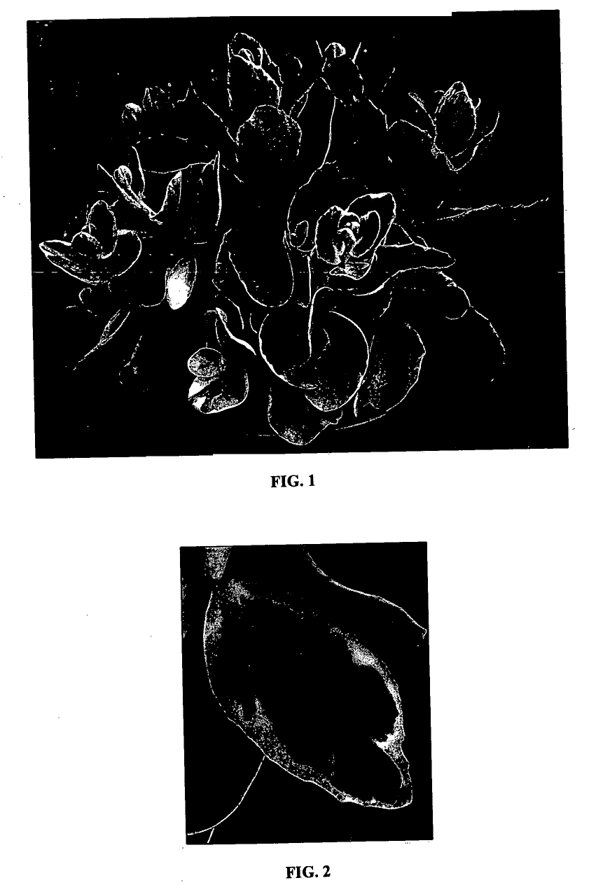

### DESCRIPTION OF THE NEW CULTIVAR

The following detailed description sets forth the distinctive characteristics of ‘Diamond Edge’. The data which define these characteristics were collected from asexual reproductions carried out in De Hoef, The Netherlands. The plant history was taken partly on two-year old plants, in June 2006. Rooted cuttings were planted in 23-cm pots and grown outdoors in De Hoef, The Netherlands. Color readings were taken under natural light. Color references are primarily to the R.H.S. Colour Chart of The Royal Horticultural Society of London (RHS) (2001).

### DETAILED BOTANICAL DESCRIPTION

Classification:
   Family.—Crassulaceae.
   Botanical.—Sedum.
   Common name.—Sedum.
Parentage: The new cultivar originated from a naturally occurring mutation in the proprietary Sedum plant ‘Matrona’ (unpatented)
Growth:
   Form.—Upright.
   Height (measured from the top of the soil).—24 cm.
   Width.—36.5 cm.
   Propagation.—Terminal tips for cutting.
   Time to produce a finished flowering plant.—24 weeks for field grown.
   Time to initiate and develop roots (March/April).—40 days.
   Root description.—RHS 155A (white), not very dense, slightly fibrous.
Leaves:
   Arrangement.—Opposite.
   Immature leaf.—Color: Upper surface: RHS 137A and RHS 137B (green) Margin (0.4 cm): RHS 151C and RHS 151D (yellow-green) Top: Darker, RHS 153D Base: Greener, RHS 145A to RHS 145B Lower surface: RHS 191A to RHS 191B (greyed-green) Margin (0.4 cm): RHS 151D (yellow-green) Top: Darker, RHS 153D Base: Greener, RHS 145A to RHS 145B Length: 4.1 cm Width: 2.6 cm.
   Mature leaf.—Color: Upper surface: RHS 137B (green) Margins (0.3 cm): RHS 151B to RHS 151C (yellow-green) Top: Darker, RHS 153C to RHS 153D, slightly tinged RHS 183B to RHS 183C (greyed-red) Base: RHS 137B to RHS 137C Lower surface: RHS 191B (greyed-green) Margins (0.3 cm): RHS 151D (yellow-green), slightly tinged RHS 183B to RHS 183C (greyed-red) Length: 11.7 cm Width: 7.7 cm.
   Apex description.—Bluntly acute to rounded.
   Base description.—Cuneate.
   Shape.—Obovate to broadly obovate, slightly curved and carinate.
   Margin.—Irregularly dentate Teeth per cm: 1.5 teeth Length of teeth: 0.2 cm.
   Texture.—Upper surface: Smooth, only slightly glossy Lower surface: Smooth, only slightly glossy.
   Venation pattern.—Pinnate.
   Venation color.—RHS 183A to RHS 183B (greyed-red), RHS 145B to RHS 145C (yellow-green) towards the base.
Leaf buds:
   Length.—0.4 cm.
   Diameter.—0.22 cm.
   Shape.—Oblong.
   Color.—RHS 145A to RHS 145B (yellow-green); tipped RHS 183D (greyed-red).
   Texture.—Smooth, dull.
Stems:
   Length.—21.9 cm.
   Internode length.—2.9 cm.
   Color.—RHS 144A to RHS 144B (yellow-green); lighter RHS 145C to RHS 145D towards the top, usually with a narrow (0.03 cm) RHS 183C to RHS 183D (greyed-purple) stripe.
   Texture.—Smooth, dull.
Fruit and seed set: None observed.
Disease and insect resistance: No special observations made.

### COMPARISON WITH PARENTAL AND COMMERCIAL CULTIVARS

‘Diamond Edge’ differs from its parent, ‘Matrona’ (unpatented) by having greenish-yellow variegated foliage, whereas ‘Matrona’ has entirely red to brownish green leaves.

‘Diamond Edge’ differs from ‘Frosty Morn’ (unpatented) by having greenish-yellow variegated foliage, while ‘Frosty Morn’ has white variegated foliage. In addition, ‘Diamond Edge’ is more stable in its variegation than ‘Frosty Morn’, which easily reverts.

I claim:

1. A new and distinct cultivar of Sedum plant as shown and described herein.
